(19) United States
(12) Patent Application Publication
(10) Pub. No.: US 2026/0085091 A1
(43) Pub. Date: Mar. 26, 2026

(54) PEPTIDES THAT SELECTIVELY BIND TO Trop2 AND USE THEREOF

(71) Applicants: KYUNGPOOK NATIONAL UNIVERSITY INDUSTRY-ACADEMIC COOPERATION FOUNDATION, Daegu (KR); MAHIDOL UNIVERSITY, Nakhon Pathom (TH)

(72) Inventors: Byung-Heon LEE, Daegu (KR); Uttapol PERMPOON, Daegu (KR); Peti Thuwajit, Bangkok (TH)

(73) Assignees: KYUNGPOOK NATIONAL UNIVERSITY INDUSTRY-ACADEMIC COOPERATION FOUNDATION, Daegu (KR); MAHIDOL UNIVERSITY, Nakhon Pathom (TH)

(21) Appl. No.: 19/107,750

(22) PCT Filed: Aug. 10, 2023
(86) PCT No.: PCT/KR2023/011883

§ 371 Date: Feb. 28, 2025

(30) Foreign Application Priority Data
Aug. 31, 2022 (KR) 10-2022-0109632

(51) Int. Cl. C07K 7/06; A61K 38/00; A61P 35/00; G01N 33/574

(52) U.S. Cl. CPC C07K 7/06; A61P 35/00; G01N 33/5758; A61K 38/00; C07K 2319/00; G01N 2333/705

(57) ABSTRACT

The present disclosure relates to a peptide binding to Trop 2 and a use thereof, wherein, in an attempt to find a peptide having an ability of specifically binding to Trop 2, a peptide (amino acid sequence: CSTLNVESC (SEQ ID NO: 1)) that selectively binds to Trop 2 was discovered using a phage display technique. Targeting cancers that are highly resistant to anticancer drugs in breast cancer (in particular, triple-negative breast cancer), pancreatic cancer, and cholangiocarcinoma while having no effective treatments, an effective anticancer drug may be prepared by delivering cancer-specific drugs or developing targeted therapeutic agents using the peptide of the present disclosure.

### CROSS REFERENCE TO PRIOR APPLICATIONS

This application is a National Stage Application of PCT International Patent Application No. PCT/KR2023/011883 filed on Aug. 10, 2023, under 35 U.S.C. § 371, which claims priority to Korean Patent Application No. 10-2022-0109632 filed on Aug. 31, 2022, which are all hereby incorporated by reference in their entirety.

### SEQUENCE LISTING

This application contains a Sequence Listing submitted via EFS-Web and hereby incorporated by reference in its entirety. The Sequence Listing is named SEQCRF 22800-564.xml, created on Feb. 26, 2025, and 5,399 bytes in size.

### TECHNICAL FIELD

The present disclosure relates to a peptide that binds to trophoblast antigen 2 (Trop2) and a use thereof.

### BACKGROUND ART

Trophoblast antigen 2 (Trop2), a membrane glycoprotein, is also defined as tumor-associated calcium signal transducer 2 (tacstd2), a membrane component surface marker-1 (M1S1), an epithelial glycoprotein-1 (EGP1), and a gastrointestinal antigen 733-1 (GA733-1). It is composed of 323 amino acids, 35-49 kDa, and includes a long extracellular domain (ECD), a single transmembrane, and a short intracellular domain (ICD). It shares high similarity with epithelial cell adhesion molecule (EpCAM), known as tacstd1 or Trop1, in terms of protein sequences and crystal structure, however, they are independent in the downstream signaling pathway. The extracellular domain of Trop2 conserves a cysteine-rich region which includes a thyroglobulin type-1 (TY) repeating domain and a putative epidermal growth factor-like domain which possibly binds to growth factors, such as insulin-like growth factor types I and II. The intracellular domain includes a sequence similar to HIKE in which a serine residue (S303) may be phosphorylated by protein kinase C and also a binding site of phosphatidylinositol 4,5-bisphosphate.

Trop2 plays an important role in the intracellular signal transduction in cancers. For instance, increasing the level of Trop2 in cervical cancer cells may induce phosphorylation of mitogen-activated protein kinases supporting cell proliferation and invasion. Modulation of beta 1 integrin by Trop2 may promote prostate cancer progression. Moreover, Src tyrosine kinase-induced nuclear accumulation of Trop2 ICD and cyclin D1 correlated with progression and poor prognosis in prostate cancer. Silencing Trop2 in gallbladder cancer cells suggested that Trop2 regulation of the PI3K/AKT pathway may induce epithelial-mesenchymal transition (EMT) and metastasis.

Cholangiocarcinoma (CCA) is an aggressive malignancy in bile duct systems which are composed of intrahepatic (ICC) and extrahepatic (ECC) CCA tissues. CCA is a rare disease globally, but there is a positive increase trend in this decade. It commonly spreads, however, in some particular regions East Asia related to liver-fluke infections which are a major risk factor in China, Japan, South Korea, and Thailand. Other chronic liver diseases are more correlated to CCA in western. A recent report showed resectable ICC represented 32.3% and 8.4% for 5-year and 10-year survival rate. Complications of late detection and limitations in treatment including drug resistance are the main issues. A study in hilar CCA, a subtype of ECC, demonstrated positive correlations of Trop2 with micro-vessel density and poor prognosis. Liver-fluke associated ICC patient tissues revealed that over 60% had a strong expression of Trop2.

The affinity peptide is an alternative option of antibodies for tumor targeting. A small peptide has advantages comparatively rather than antibody in the case of deep tissue penetration, low immunogenicity, and simple modification, however, a short circulation time in the blood stream is a limitation. Biopanning using peptides displaying T7 phage libraries are widely used to identify surface antigen targeting-peptides. Peptide library which displays CX7C (C, cysteine; X, any amino acid residue) on the capsid of T7 phages was constructed. This phage display established an interleukin-4 receptor alpha (IL-4Rα)-binding peptide, IL4RPep-1, which provided effective tumor targeting and cellular internalization. The conjugation with proapoptotic peptide (KLAKLAK (SEQ ID NO: 3)) 2 exhibited not only an anti-tumor effect but also turned M2-polarized tumor associated macrophages (TAM) to M1-polarized macrophages.

As described above, the phage display of the peptide is a very useful method for identifying ligands specific to target cells and is widely used to discover peptides targeting cancer cells in vitro and in vivo. However, peptides targeting Trop2 or drug delivery technologies and targeted therapies using the same have not yet been reported.

### DISCLOSURE OF INVENTION

### Technical Problem

The present disclosure relates to a peptide binding to Trop 2 and a use thereof, and an object of the present disclosure is to provide a peptide which has an amino acid sequence represented by SEQ ID NO: 1 and specifically binds to Trop 2, a composition for cancer diagnosis, a composition for drug delivery, a composition for cancer cell imaging including the peptide as an active ingredient, a fusion peptide in which the peptide, a cell-permeable peptide having an amino acid sequence represented by SEQ ID NO: 2, and a proapoptotic peptide having an amino acid sequence represented by SEQ ID NO: 3 are combined, and a pharmaceutical composition for preventing or treating cancer including the fusion peptide as an active ingredient.

### Solution to Problem

To solve the above problem, the present disclosure provides a peptide which has an amino acid sequence represented by SEQ ID NO: 1 and specifically binds to Trop 2.

In addition, the present disclosure provides a polynucleotide encoding the peptide, a recombinant vector including the polynucleotide, and a transformant transformed by the recombinant vector.

In addition, the present disclosure provides a composition for cancer diagnosis, including the peptide as an active ingredient.

In addition, the present disclosure provides a composition for drug delivery, including the peptide as an active ingredient.

In addition, the present disclosure provides a fusion peptide in which the peptide, a cell-permeable peptide having an amino acid sequence represented by SEQ ID NO: 2, and a proapoptotic peptide having an amino acid sequence represented by SEQ ID NO: 3 are combined.

In addition, the present disclosure provides a composition for cancer cell imaging, including the peptide as an active ingredient.

### Advantageous Effects of Invention

The present disclosure relates to a peptide binding to Trop 2 and a use thereof, wherein, in an attempt to find a peptide having a binding ability specific to Trop 2, a peptide (amino acid sequence: CSTLNVESC (SEQ ID NO: 1)) that selectively binds to Trop 2 was discovered using a phage display technique. Targeting cancers that are highly resistant to anticancer drugs in breast cancer (in particular, triple-negative breast cancer), pancreatic cancer, and cholangiocarcinoma while having no effective treatments, an effective anticancer drug may be prepared by delivering cancer-specific drugs or developing targeted therapeutic agents using the peptide of the present disclosure.

### BRIEF DESCRIPTION OF DRAWINGS

FIG. 1 shows results of Trop 2 expression in cholangiocarcinoma cell lines.

FIG. 2 shows results of Trop 2 expression in pcDNA-Trop 2-transfected HEK293T cells.

FIG. 3 shows biopanning results of phage library using Trop2-overexpressing cells.

FIG. 4 shows results of detecting the binding of a CSTLNVESC (SEQ ID NO: 1) peptide to a Trop 2 protein.

FIGS. 5 and 6 show results of detecting the binding of a CSTLNVESC (SEQ ID NO: 1) peptide to Trop2-overexpressing cells.

FIG. 7 shows results of measuring cell binding affinity using a ligand tracer.

FIG. 8 shows results on cytotoxicity of a CSTLNVESC (SEQ ID NO: 1) peptide in a cholangiocarcinoma cell line.

FIG. 9 shows results of serum stability analysis.

FIG. 10 shows results of identifying inhibition of cell migration and invasion by a CSTLNVESC (SEQ ID NO: 1) peptide.

FIG. 11 shows results on tumor homing of a CSTLNVESC (SEQ ID NO: 1) peptide.

FIG. 12 shows results on an antitumor growth activity of a CSTLNVESC (SEQ ID NO: 1)-targeted cytotoxic peptide.

### BEST MODE FOR CARRYING OUT THE INVENTION

The present disclosure provides a peptide which has an amino acid sequence represented by SEQ ID NO: 1 and specifically binds to trophoblast antigen 2 (Trop2).

The peptide of the present disclosure may be easily prepared by chemical synthesis known in the art (Creighton, Proteins; Structures and Molecular Principles, W. H. Freeman and Co., NY, 1983). Representative methods include liquid or solid phase synthesis, fragment condensation, F-MOC, or T-BOC chemical methods (Chemical Approaches to the Synthesis of Peptides and Proteins, Williams et al., Eds., CRC Press, Boca Raton Florida, 1997; A Practical Approach, Athert on & Sheppard, Eds., IRL Press, Oxford, England, 1989), but are not limited thereto.

In addition, the peptide of the present disclosure may be prepared by a genetic engineering method. First, a DNA sequence encoding the peptide is synthesized according to a conventional method. DNA sequences may be synthesized by PCR amplification using appropriate primers. DNA sequences may be synthesized by other standard methods known in the art, for example, using an automatic DNA synthesizer (e.g., those sold by Biosearch or Applied Biosystems). The prepared DNA sequence is inserted into a vector including one or more expression control sequences (e.g., promoter, enhancer, etc.) that are operatively linked to the DNA sequence and regulate expression of the DNA sequence, so as to transform a host cell with the recombinant expression vector formed therefrom. The resulting transformants are cultured under appropriate media and conditions to allow expression of the DNA sequence to harvest substantially pure peptides encoded by the DNA sequence from the culture. The harvest may be performed using a method known in the art (e.g., chromatography). The term ‘substantially pure peptide’ as used herein refers to a state that the peptide according to the present disclosure does not substantially include any other proteins derived from the host.

In the present disclosure, the peptide having an amino acid sequence represented by SEQ ID NO: 1 is a concept including a functional variant thereof. The term “functional variant” as used herein refers to all similar sequences in which substitutions of some amino acids occur at an amino acid locus without affecting the properties of the peptides of the present disclosure that specifically bind to Trop2.

In addition, the present disclosure provides a polynucleotide encoding the peptide.

The term “polynucleotide” as used herein refers to a polymer of deoxyribonucleotides or ribonucleotides that exist in single-stranded or double-stranded form. The polynucleotide includes RNA genomic sequences, DNA (gDNA and cDNA), and RNA sequences transcribed therefrom and also includes analogs of natural polynucleotides unless otherwise specified.

The polynucleotide includes not only the nucleotide sequence encoding the peptide, but also a sequence complementary to the sequence. The complementary sequence includes not only perfectly complementary sequences, but also substantially complementary sequences.

In addition, the polynucleotide may be modified. Such modifications include additions, deletions or non-conservative substitutions or conservative substitutions of nucleotides. The polynucleotide encoding the amino acid sequence is construed to include a nucleotide sequence showing substantial identity to the nucleotide sequence. The substantial identity may refer to, when the nucleotide sequence and any other sequence are aligned to the maximum correspondence and the aligned sequence is analyzed using an algorithm commonly used in the art, a sequence showing at least 80% homology, at least 90% homology or at least 95% homology.

In addition, the present disclosure provides a recombinant vector including the polynucleotide.

In addition, the present disclosure provides a transformant (provided that humans are excluded) transformed by the recombinant vector.

The term “vector” as used herein refers to a self-replicating DNA molecule used to carry a clonal gene (or another fragment of clonal DNA).

The term “recombinant vector” as used herein may refer to a plasmid, viral vector or other media known in the art capable of expressing the inserted nucleic acid in a host cell, and one that polynucleotides encoding the peptides of the present disclosure are operably linked to a conventional expression vector known in the art. The recombinant vector may include an origin of replication to generally enable proliferation in a host cell, and one or more expression regulatory sequences (e.g., promoter, enhancer, etc.) to regulate expression, a selective marker, and a polynucleotide encoding a peptide of the present disclosure operably linked to an expression regulatory sequence. The transformant may be transformed by the recombinant vector.

Preferably, the transformant may be obtained by introducing a recombinant vector including a polynucleotide encoding the peptide of the present disclosure into a host cell by a method known in the art, for example, but not limited to, transient transfection, microinjection, transduction, cell fusion, calcium phosphate precipitation, liposome-mediated transfection, DEAE dextran-mediated transfection, polybrene-mediated transfection, electroporation, gene gun, and other known methods to introduce a nucleic acid into the cell (Wu et al., J. Bio. Chem., 267:963-967, 1992; Wu and Wu, J. Bio. Chem., 263:14621-14624, 1988).

In addition, the present disclosure provides a composition for cancer diagnosis, including the peptide as an active ingredient.

Preferably, the cancer may be a cancer with a high expression level of Trop 2, but is not limited thereto.

More preferably, the cancers may be breast cancer (in particular, triple-negative breast cancer), lung cancer, pancreatic cancer, brain tumor, liver cancer, esophageal cancer, kidney cancer, colorectal cancer, rectal cancer, stomach cancer, thyroid cancer, bladder cancer, ovarian cancer, cholangiocarcinoma, bile duct cancer, gallbladder cancer, uterine cancer, cervical cancer, prostate cancer, head and neck cancer, laryngeal cancer, and oral cavity cancer, but are not limited thereto.

The term “diagnosis” as used herein refers to identification of the presence or characteristics of a pathological condition. For the purposes of the present disclosure, diagnosis is to identify the presence or characteristics of cancer.

Diagnosis of cancer using the peptide of the present disclosure may be diagnosed by reacting the peptide of the present disclosure with the tissue or cell obtained directly from blood, urine, or biopsy, followed by detection of the binding thereof.

In addition, in order to easily identify, detect and quantify whether the peptide of the present disclosure binds to cancer tissues, the peptide of the present disclosure may be provided in a labeled state. In other words, it may be provided by being linked (e.g., covalently bonded or cross-linked) to a detectable label. The detectable label may be a chromogenic enzyme (e.g., peroxidase, alkaline phosphatase), a radioactive isotope (e.g., ¹²⁴I, ¹²⁵I, ¹¹¹In, ⁹⁹mTc, ³²P, ³⁵S), a chromophore, a luminescent material or a fluorescer (e.g., FITC, RITC, rhodamine, cyanine, Texas Red, fluorescein, phycoerythrin, and quantum dots).

Similarly, the detectable label may be an antibody epitope, a substrate, a cofactor, an inhibitor, or an affinity ligand. Such labeling may be performed during the process of synthesizing the peptide of the present disclosure or may be additionally performed on the pre-synthesized peptide. If the fluorescer is used as a detectable label, cancer may be diagnosed by fluorescence mediated tomography (FMT). For example, the peptide of the present disclosure labeled with the fluorescer may be circulated in the blood and the fluorescence by the peptide may be observed via fluorescence tomography. If fluorescence is observed, cancer is diagnosed.

In addition, the present disclosure provides a composition for drug delivery, including the peptide as an active ingredient.

Preferably, the drug may be a peptide drug or an anticancer agent, and more preferably, the peptide drug may be a cytotoxic peptide having a proapoptotic or pronecrotic activity, but is not limited thereto.

The peptide according to the present disclosure may be used as an intelligent drug carrier to selectively deliver drugs to cancer cells. If the peptide of the present disclosure is used for cancer treatment in connection with a previously known drug, the effect of the drug may be increased because the drug is selectively delivered to cancer cell-derived exosomes by the peptide of the present disclosure, and at the same time, the side effects of the drug on normal tissues may be significantly reduced.

The drug is an anticancer drug, and as the anticancer drug that may be linked to the peptide of the present disclosure, there is no limit as long as it is conventionally used for the treatment of cancer. For example, the drug may be mertansine, doxorubicin, paclitaxel, vincristine, daunorubicin, vinblastine, actinomycin-D, docetaxel, etoposide, teniposide, bisantrene, homoharringtonine, Gleevec (STI-571), cisplatin, 5-fluorouracil, adriamycin, methotrexate, busulfan, chlorambucil, cyclophosphamide, melphalan, nitrogen mustard, and nitrosourea. The linkage between the anticancer drug and the peptide of the present disclosure may be performed by methods known in the art, for example, covalent bonding and crosslinking. To this end, if necessary, the peptide of the present disclosure may be chemically modified in a range in which the activity thereof is not lost.

In addition, the present disclosure provides a fusion peptide in which the peptide, a cell-permeable peptide having an amino acid sequence represented by SEQ ID NO: 2, and a proapoptotic peptide having an amino acid sequence represented by SEQ ID NO: 3 are combined.

The cell-permeable peptide having the amino acid sequence represented by SEQ ID NO: 2 is “RRRRRRR”, abbreviated herein as “R7”.

The proapoptotic peptide having the amino acid sequence represented by SEQ ID NO: 3 is “KLAKLAKKLAKLAK (SEQ ID NO: 3)” or “(KLAKLAK (SEQ ID NO: 3)) 2”, abbreviated herein as “KLA”.

In addition, the present disclosure provides a pharmaceutical composition for preventing or treating cancer, including the fusion peptide as an active ingredient.

Preferably, the cancer may be a cancer with a high expression level of Trop 2, but is not limited thereto.

More preferably, the cancers may be breast cancer (in particular, triple-negative breast cancer), lung cancer, pancreatic cancer, brain tumor, liver cancer, esophageal cancer, kidney cancer, colorectal cancer, rectal cancer, stomach cancer, thyroid cancer, bladder cancer, ovarian cancer, cholangiocarcinoma, bile duct cancer, gallbladder cancer, uterine cancer, cervical cancer, prostate cancer, head and neck cancer, laryngeal cancer, and oral cavity cancer, but are not limited thereto.

The pharmaceutical composition of the present disclosure may be prepared using a pharmaceutically suitable and physiologically acceptable adjuvant in addition to the active ingredient, and a solubilizing agent such as an excipient, a disintegrant, a sweetener, a binder, a coating agent, a swelling agent, a lubricant, a polishing agent, or a flavoring agent may be used as the adjuvant. The pharmaceutical composition of the present disclosure may be preferably formulated into a pharmaceutical composition by including one or more pharmaceutically acceptable carriers in addition to the active ingredient for administration. In the composition formulated as a liquid solution, pharmaceutically acceptable carriers may be sterile and biocompatible and used by mixing saline, sterile water, Ringer's solution, buffered saline, albumin injection, dextrose solution, maltodextrin solution, glycerol, ethanol and one or more of these components, while other conventional additives such as antioxidants, buffers, and bacteriostats may be added as needed. In addition, diluents, dispersants, surfactants, binders, and lubricants may be additionally added to prepare into an injectable formulation such as an aqueous solution, suspension, and emulsion as well as pills, capsules, granules, or tablets.

The pharmaceutical formulation form of the pharmaceutical composition of the present disclosure may be granules, powder, coated tablets, tablets, capsules, suppositories, syrups, juices, suspensions, emulsions, drops or injectable solutions, and sustained-release formulations of the active compound. The pharmaceutical composition of the present disclosure may be administered in a conventional manner via intravenous, intraarterial, intraperitoneal, intramuscular, intraarterial, intraperitoneal, intrasternal, transdermal, intranasal, inhalational, topical, rectal, oral, intraocular or intradermal routes. The effective amount of the active ingredient of the pharmaceutical composition of the present disclosure refers to an amount required for preventing or treating a disease. Therefore, the effective amount may be controlled by various factors including the type of disease, the severity of the disease, the type and content of the active ingredient and other ingredients included in the composition, the type of formulation and the age, weight, general health status, sex and diet of a patient, administration time, administration route and secretion rate of the composition, treatment period, and drugs used in combination with. For example, although not limited thereto, in the case of adults, when administered once to several times a day, the composition of the present disclosure may be administered at a dose of 0.1 ng/kg to 10 g/kg in case of a compound, when administered once a day to several times.

In addition, the present disclosure provides a composition for cancer cell imaging, including the peptide as an active ingredient.

Preferably, the peptide may be labeled with chromogenic enzymes, radioactive isotopes, chromophores, luminescent materials, fluorescers, magnetic resonance imaging materials, superparamagnetic particles, or ultra superparamagnetic particles, but is not limited thereto.

Cancer cell imaging and cancer diagnosis are not limited thereto but may be used not only for the initial examination of cancer diseases, but also for progression, treatment progress for treatment, and monitoring of response to therapeutic agents. The peptide may be provided in a labeled state to facilitate identification, detection, and quantification of binding status, as described above.

### MODE FOR THE INVENTION

Hereinafter, the present disclosure will be described in detail according to an example embodiment that does not limit the present disclosure. It is obvious that the following example embodiments of the present disclosure are only for the purpose of embodying the present disclosure and do not restrict or limit the scope of rights of the present disclosure. Therefore, what may be easily inferred by an expert in the art to which the present disclosure pertains from the detailed description and example embodiments of the present disclosure is interpreted as belonging to the scope of rights of the present disclosure.

### EXPERIMENTAL EXAMPLE

The following experimental examples are intended to provide experimental examples commonly applied to each example embodiment according to the present disclosure.

### 1. Cell Cultures

KKU-213 and KKU-055 human intrahepatic cholangiocarcinoma cell lines were purchased from the Japanese Collection of Research Bioresources Cell Bank (JCRB Cell Bank, Tokyo, Japan). HEK293T human embryonic kidney cells were purchased from ATCC (Manassas, VA, USA). KU-213, KKU-055, and HEK293T cells were cultured in Dulbecco Modified Eagle Medium (DMEM) with 10% fetal bovine serum (FBS), 100 U/ml penicillin, and 100 μg/ml streptomycin at 37° C. with 5% carbon dioxide (CO₂).

To prepare Trop2-overexpressing cells for biopanning, pCMV6-AC-GFP plasmid including human Trop2 gene, tacstd2, (Origene, Rockville, MD, USA) was transfected into HEK293T cells using Lipofectamine 2000 (Thermo Fisher Scientific, Waltham, MA, USA). For Trop2 silencing, KKU-213 cells were transfected with a small-interfering RNA (siRNA) against Trop2 gene (Qiagen, Venlo, Netherlands) or control siRNA (Bioneer, Oakland, CA) using Lipofectamine RNAiMAX (Thermo Fisher Scientific). Two siRNAs, siTrop21 and siTrop22 have been designed specifically to target sequences; 5′-CCC GGG ATC GTT TGC AAG TAA-3 (SEQ ID NO: 4)′ and 5′-TTG CGA CAT TGT GAA GGC TTA-3 (SEQ ID NO: 5)′. The silencing efficiencies were analyzed by Western blots.

### 2. Biopanning of T7 Phage Library and Selection of Trop2-Binding Peptides

A phage peptide library based on T7 415-1b phage vector displaying CX7C (C, cysteine; X, any amino acid residue) were constructed according to the manufacturer's manual (Novagen, Madison, WI). The library had a diversity of 5×10⁸ plaque-forming units (pfu). The DNA insert of selected phage clones was sequenced by Koma Biotech Co. (Daejeon, Republic of Korea). For biopanning, Trop2-transfected HEK293T cells were cultured in 35-mm dishes. The cells were blocked with culture medium including 1% bovine serum albumin (BSA) at room temperature (RT) for 30 min and reacted with the T7 phage library (approximately 1×10⁹ pfu) at 4° C. for 1 h. The unbound phages were removed, and cells were washed with phosphate buffered saline (PBS) for 3 times. The binding phages were eluted with E. coli BL21 (OD₆₀₀ 1.0)-enriched Luria-Bertani (LB) broth at RT for 10 min. The eluted phages were amplified with E. coli BL21 (OD₆₀₀ 0.5)-enriched LB broth at 37° C. for 1-3 h or until lysis was observed. The titers of the amplified phages were determined by a plague formation assay, and the phages were used for next round. The eluted phages from the third and fourth round were randomly selected, amplified, and analyzed for their peptide sequences.

All peptides were synthesized by the standard fluorenyl methoxycarbonyl protecting group (Fmoc) method using a peptide synthesizer and purified by high-performance liquid chromatography (HPLC) to >90% purity, and their masses were confirmed by matrix assisted laser desorption ionization-time of flight (Peptron Inc., Daejeon, Republic of Korea). Peptides were conjugated at the N-terminal with biotin. For fluorescence labeling, peptides were conjugated at the C-terminal with fluorescein isothiocyanate (FITC), or with near-infrared Flamma® 675 dye (Bioacts, Incheon, Republic of Korea). The NSSSVDK peptide, a sequence present in T7 phage coat protein, was used as a negative control.

### 3. Enzyme-Linked Immunosorbent Assay (ELISA) for Cell Binding of Phage and Protein Binding of Peptide

Cells seeded in a 96-well plate were blocked with 1% BSA at RT for 30 min, then reacted with each phage clone (1×10⁹ pfu) at RT for 1 h. The plates were washed with PBS before reaction with mouse anti-T7 monoclonal antibody (Novagen) at RT for 1 h and were reacted with horseradish peroxidase (HRP)-conjugated goat anti-mouse IgG monoclonal antibody (Santa Cruz Biotechnology, Dallas, TX, USA) at RT for 1 h. After washing with PBS, 1-Step™ Turbo TMB-ELISA substrate solution was added (Thermo Fisher Scientific), and plates were reacted at RT for 10-30 min followed by addition of 2N H₂SO₄ stop solution. The absorbance was measured at 450 nm with a microplate reader.

Recombinant Trop2 (rTrop2) or BSA (0.5 μg in 100 μl solution) were coated on ELISA microplates with 0.1 M NaHCO₃(pH 7.6) at 4° C. overnight. The plate was washed with PBS and blocked with 1% BSA at RT for 1 h. The biotinylated peptide (1 μg in 100 μl solution) was added to the protein-coated plates and reacted at RT for 1 h and with HRP-conjugated streptavidin at RT for 1 h. After washing with PBS several times, 100 μl of substrate solution was added. A stop solution was added after a blue color appeared. The absorbance was measured at 450 nm with a microplate reader.

### 4. Immunofluorescence Analysis

Cells were seeded on 4-well chamber slides and kept at 37° C. overnight. The cells were gently cleaned with PBS and fixed with 4% paraformaldehyde (PFA) at RT for 10 min. After blocking with 1% BSA for 30 min, slides were reacted with mouse anti-Trop2 monoclonal antibody (Abcam, Cambridge, UK) at 4° C. overnight. Then, cells were reacted with Alexa Fluor 488-labeled anti-mouse IgG antibody (Thermo Fisher Scientific) at RT for 1 h, and counter-stained with 4′,6-diamidino-2-phenylindole dihydrochloride (DAPI) (Dojindo, Kumamoto, Japan) at RT for 10 min. Slides were enclosed with anti-fade solution and covered up with a cover glass. The slides were analyzed with a fluorescence microscope.

For co-localization assays, cells were reacted with FITC-conjugated peptide at 4° C. for 1 h and fixed with 4% PFA at RT for 10 min. Next, cells were reacted with mouse anti-Trop2 antibody at 4° C. overnight and then with Alexa Fluor 594-labeled anti-mouse IgG antibody (Thermo Fisher Scientific) at RT for 1 h. Cells were counter-stained with DAPI and enclosed with an anti-fade agent. The slides were examined using a confocal microscope.

For tissue staining, frozen tissues on glass slides were reacted with 0.3% Triton X-100/PBS at RT for 15 min and cleaned with PBS. After blocking with 1% BSA/PBS at RT for 30 min, tissue slides were reacted with rabbit anti-Trop2 antibody (Abcam) at 4° C. overnight and then with Alexa Fluor 488-labeled anti-rabbit IgG antibody (Thermo Fisher Scientific) at RT for 1 h. Tissue slides were reacted with DAPI at RT for 10 min and enclosed.

### 5. Flow Cytometry Analysis

A million cells were prepared, fixed with 4% PFA, and blocked with 1% BSA at RT for 30 min. The cells were reacted with mouse anti-Trop2 antibody at RT for 1 h with agitation. Then, cells were reacted with Alexa Fluor 488-labeled anti-mouse IgG antibody at RT for 1 h. For peptide binding, cells were reacted with FITC-labeling peptide at 4° C. for 1 h with agitation. The stained cells were fixed with 4% PFA before analysis with a flow cytometer.

### 6. Western Blotting Analysis

Cell lysates were run in 10% sodium dodecyl sulfate-polyacrylamide gel (SDS-PAGE) and transferred to polyvinylidene difluoride (PVDF) membranes. After blocking with 5% skim milk, the membranes were reacted with rabbit anti-Trop2 antibody at 4° C. overnight, and then reacted with HRP-conjugated goat anti-rabbit IgG antibody (Cell Signaling Technology, Danvers, MA, USA) at RT for 1 h. Band signals were detected by luminescent image analysis system (LAS-1000, Fujifilm, Tokyo, Japan).

### 7. Immunoprecipitation Assays

Cell lysates of Trop2-overexpressing HEK293T cells were reacted with biotinylated peptide at RT for 2 h on a rotator. Excess of unlabeled peptide was added for competition assays. The biotin-linked peptide-protein complex was precipitated with monomeric avidin-labeled magnetic beads (Bioclone, San Diego, CA, USA) at RT for 1 h on the rotator. The protein complexes were eluted with elution buffer and subjected to western blotting analysis using anti-Trop2 antibody.

### 8. Migration and Invasion Assays

Serum-starved KKU-213 cells were resuspended with serum-free medium containing peptide and added to the 8.0 μm pore polycarbonate insert of a Transwell chamber (Corning Life Sciences, Tewksbury, MA, USA). For the invasion assay, the membrane was coated with 0.3 μg Matrigel matrix (Corning Life Sciences) at 37° C. for 2 h before use. Serum-containing or serum-free medium was added to the reservoir and reacted at 37° C. overnight. The migrated or invaded cells were fixed and stained with 0.1% crystal violet solution at RT for 10 min. The stained cells were counted under a light microscope.

### 9. Tumor Homing Assays

Four-week-old BALB/c female nude mice were purchased from Orient Bio (Seongnam, Republic of Korea). The mice were cared for and maintained in conformance with the Guidelines of the Institutional Animal Care and Use Committee (IACUC) of Kyungpook National University (permission no. 2017-0077). The mice were maintained in specific-pathogen free condition, controlled temperature (22±2° C.), humidity (50±10%), and light/dark cycle (12:12 h). Sterile tablet food, water and bedding were provided ad libitum. The mice were injected subcutaneously with approximately two million KKU-213 cells resuspended in 100 μl DMEM. When the tumor size reached 200 mm³ after approximately 4 weeks, the mice were intravenously injected with near-infrared Flamma Fluor 675-labeled peptides. After 2 h, the signal was detected using the In Vivo Imaging System (IVIS) (PerkinElmer, Waltham, MA, USA). During the analysis the mice were anesthetized with 2.5% isoflurane in a gas chamber supplied with oxygen flowmeter 0.8 L/min. Then, the mice were sacrificed by 30% CO₂ gas chamber euthanasia, and the signals were observed in vital organs and tumors. The frozen tissues were prepared for histological examination.

### 10. Statistical Analysis

Student t-tests and one-way ANOVA tests were used to analyze statistical significances between groups.

### <Example 1> Trop2 Expression in CCA Cell Lines

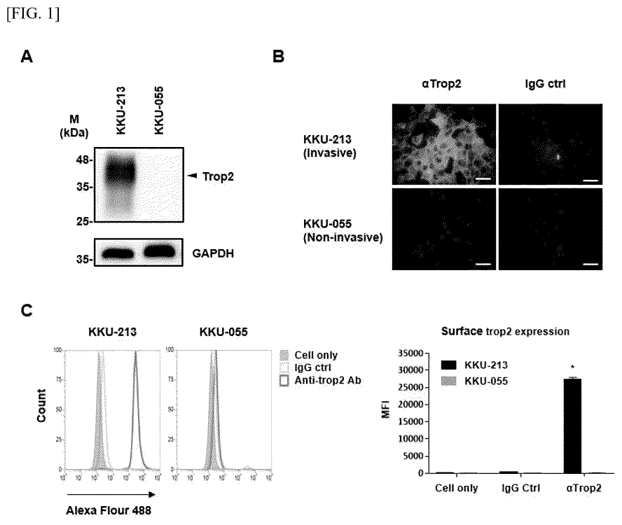

To examine Trop2 expression in KKU-213 and KKU-055 human CCA cell lines, the cell lysates were analyzed by western blotting (FIG. 1A). Trop2 was found in KKU-213 cells, but not in KKU-055 cells. Immunofluorescence (FIG. 1B) and flow cytometry (FIG. 1C) showed that almost all KKU-213 cells highly expressed Trop2 on the cell membrane, while KKU-055 cells little expressed Trop2.

### <Example 2> Biopanning for Trop2-Binding Peptides Using a T7 Phage Library

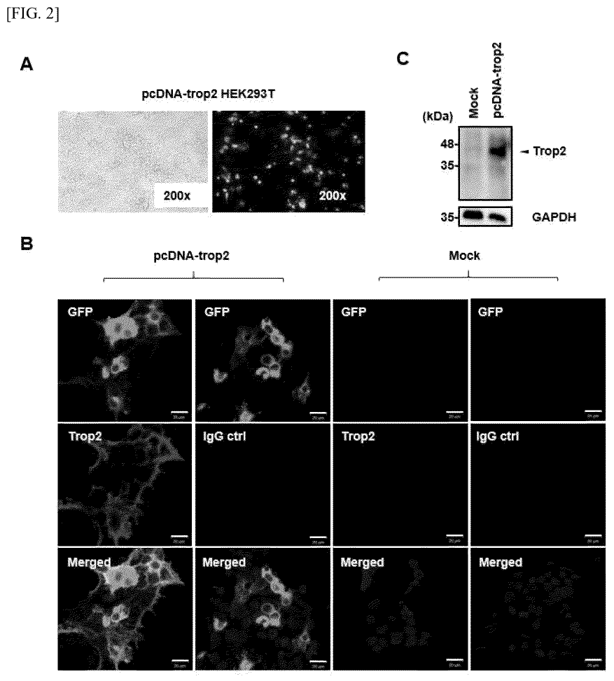

For peptide biopanning, a plasmid encoding Trop2 gene (tacstd2) linked with green fluorescence protein (GFP) was transfected into HEK293T normal embryonic kidney cells to prepare transient Trop2-overexpressing cells. Immunofluorescence microscopic analysis showed that almost cells expressed GFP protein after 48 h of transfection (FIG. 2A). Additionally, Trop2 expression was observed at the cell membrane (FIG. 2B). Western blotting showed that the transfected cells expressed a mature form (glycosylation) of Trop2 compared with mock-transfected control cells (FIG. 2C).

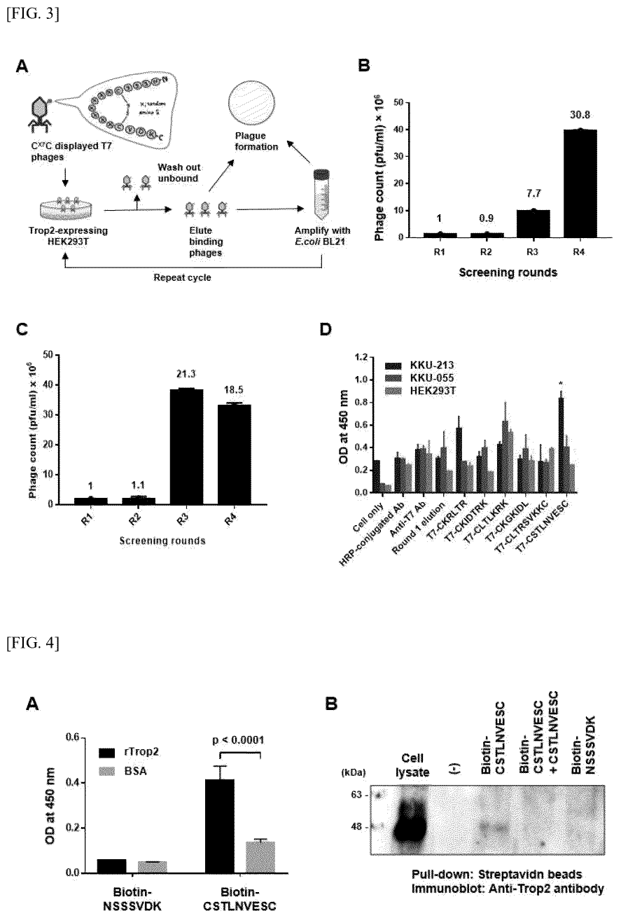

FIG. 3A schematically represents the biopanning procedures for peptides using a T7 phage library displaying random CXXXXXXXC peptides. The phage library was reacted with the transient Trop2-overexpressing HEK293T cells. The cell-bound phages were eluted and amplified for the next round of screening. Phage titers were determined by plague assays. After the third and fourth round, the phage titers were enriched by 7.7-fold and 30.8-fold, respectively (FIG. 3B). In parallel, screening was also performed against non-transfected HEK293T cells (FIG. 3C). Candidate phages were randomly picked and analyzed for their peptide-coding DNA inserts by sequencing. Thereamong, 7 candidates were from the screening against Trop2-overexpressing HEK293T cells, not from the non-transfected HEK293T cells. Candidate sequences that were shorter than 6-mer were excluded. Then, individual phage clones were amplified and analyzed for their cell binding using ELISA (FIG. 3D). Among the phage clones, a phage clone expressing CSTLNVESC (SEQ ID NO: 1) peptide bound to Trop2-positive KKU-213 cell at higher levels than to Trop2-negative KKU-055 and HEK293T cells. Subsequently CSTLNVESC (SEQ ID NO: 1) peptide was selected for further study.

### <Example 3> Binding of CSTLNVESC (SEQ ID NO: 1) Peptide to Trop2 Protein

To examine the binding of CSTLNVESC (SEQ ID NO: 1) peptide to Trop2 protein, N-terminal biotin-linked CSTLNVESC (SEQ ID NO: 1) peptide was synthesized, and binding activity of the peptide bound to the human recombinant Trop2 protein coated on an ELISA plate was detected by streptavidin-conjugated HRP (FIG. 4A). BSA was used as a control (FIG. 4A). Next, biotin-labeled peptide was reacted with the cell lysates of Trop2-overexpressing HEK293T cells and then the Trop2-peptide complexes were pulled down using streptavidin-labeled magnetic beads. Biotin-labeled CSTLNVESC (SEQ ID NO: 1) peptide bound to Trop2 of the cell lysates and precipitated, and this was inhibited by addition of unlabeled CSTLNVESC (SEQ ID NO: 1) peptide at a higher concentration (FIG. 4B).

### <Example 4> Binding of CSTLNVESC (SEQ ID NO: 1) Peptide to Trop2-Overexpressing Cells

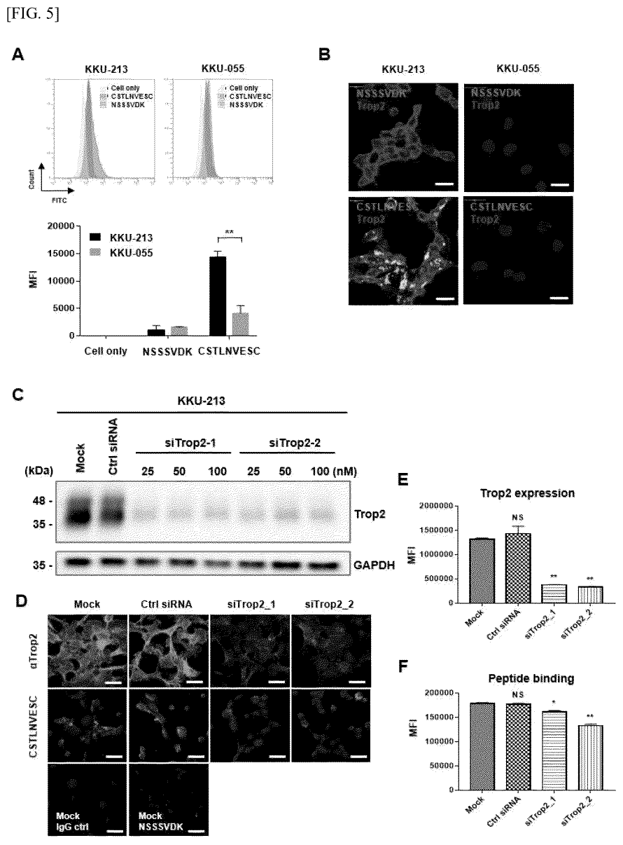

To examine the binding of CSTLNVESC (SEQ ID NO: 1) peptide to cells, CSTLNVESC (SEQ ID NO: 1) peptide was tagged with FITC fluorescence dye at the carboxy terminus. Flow cytometry showed that CSTLNVESC (SEQ ID NO: 1)-FITC peptide bound to KKU-213 cells at higher levels than to KKU-055 cells (FIG. 5A). In contrast, NSSSVDK-FITC control peptide weakly bound to both cell lines. Binding of CSTLNVESC (SEQ ID NO: 1)-FITC peptide, but not NSSSVDK-FITC peptide, to Trop2-expressing KKU-213 cells was also observed by immunofluorescence microscopic analysis (FIG. 5B).

To further examine whether the binding of CSTLNVESC (SEQ ID NO: 1)-FITC peptide to the Trop2-overexpressing cells was mediated by Trop2, Trop2 gene expression in KKU-213 cells was silenced using Trop2 siRNAs. Trop2 expression was reduced after transfection with Trop2 siRNAs for 24 h as determined by western blotting (FIG. 5C), immunofluorescence microscopy (FIG. 5D), and flow cytometry (FIG. 5E). Subsequently, the binding of CSTLNVESC (SEQ ID NO: 1)-FITC peptide to the Trop2-silenced cells was reduced (FIGS. 5D and 5F).

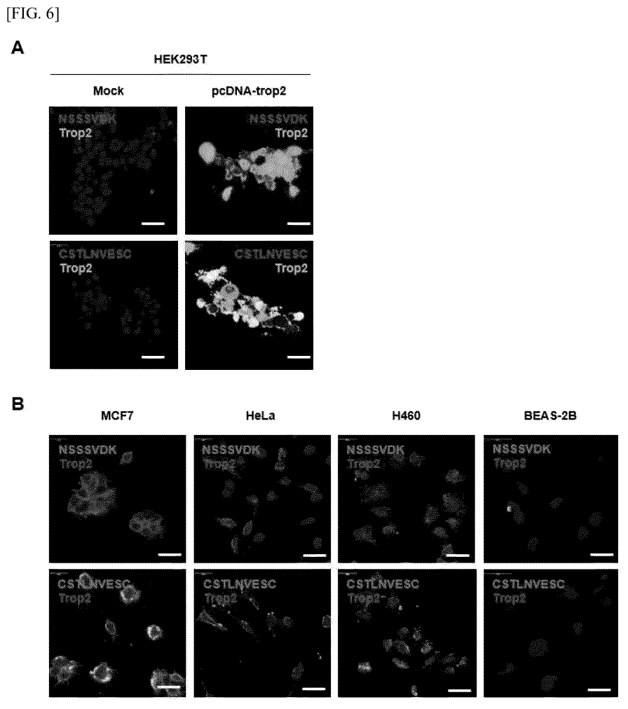

In addition, CSTLNVESC (SEQ ID NO: 1)-FITC peptide selectively bound to Trop2-overexpressing HEK293T cells but not to wild-type HEK293T cells (FIG. 6A) and Trop2-overexpressing MCF7 breast tumor, HeLa cervical tumor, and H460 lung tumor cells but not to Trop2-low BEAS-2B normal bronchial epithelial cells (FIG. 6B).

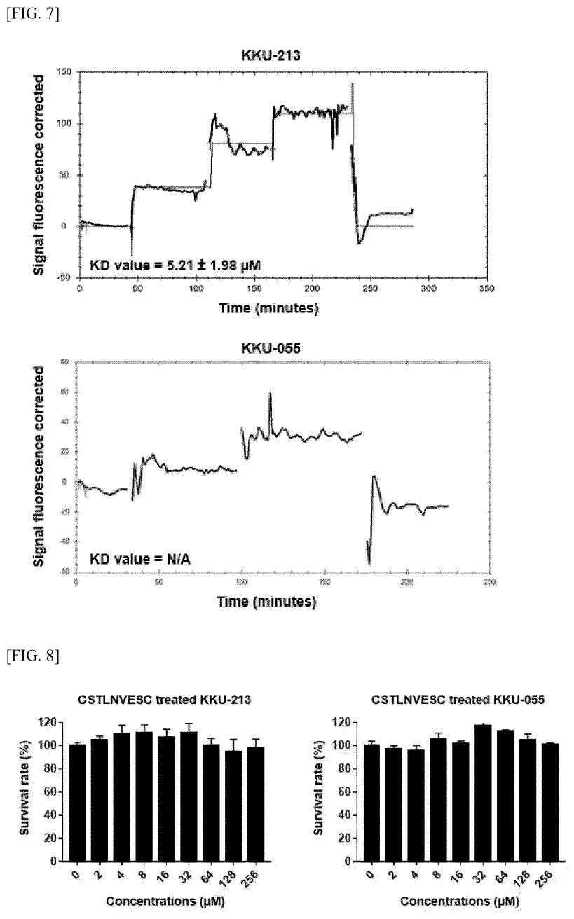

To determine the cell binding affinity of CSTLNVESC (SEQ ID NO: 1) peptide, a binding assay using the Ligand Tracer was performed. The machine continuously monitored the CSTLNVESC (SEQ ID NO: 1)-FITC peptide binding to KKU-213 or KKU-055 cells that was dose and time-dependent. The K_{D} value of the binding of CSTLNVESC (SEQ ID NO: 1)-FITC to KKU-213 cells was 5.21±1.98 μM, while its binding to KKU-055 cells was hardly detected (FIG. 7).

To examine whether CSTLNVESC (SEQ ID NO: 1) peptide affects cell survival, KKU-213 and KKU-055 cells were reacted with the peptide for 24 h. No cytotoxicity was observed both in KKU-213 and KKU-055 cells after treatment with CSTLNVESC (SEQ ID NO: 1) peptide at a concentration up to 256 μM (FIG. 8).

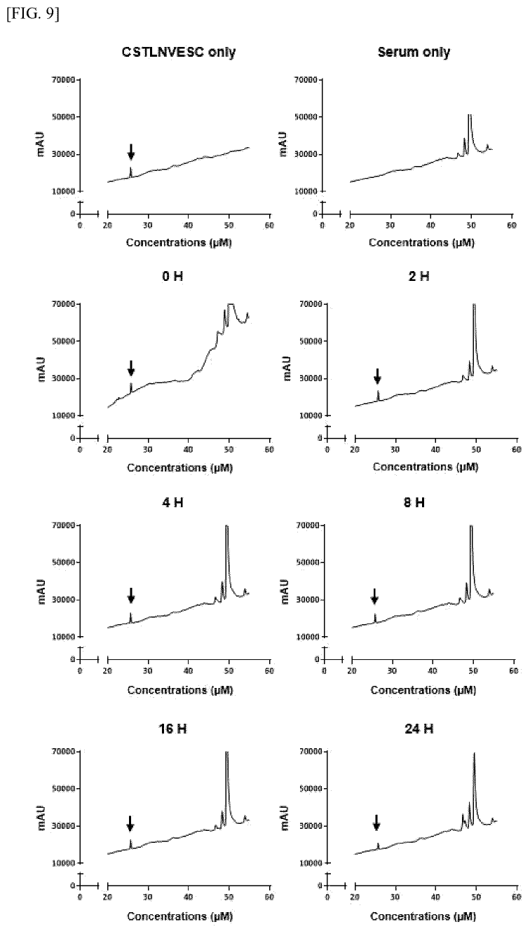

To examine the stability of CSTLNVESC (SEQ ID NO: 1) peptide, the peptide was incubated with mouse serum at 37° C. for 24 h and analyzed by HPLC. CSTLNVESC (SEQ ID NO: 1) peptide was stable until 16 h and declined thereafter (FIG. 9).

### <Example 5> Inhibition of Cell Migration and Invasion by CSTLNVESC (SEQ ID NO: 1) Peptide

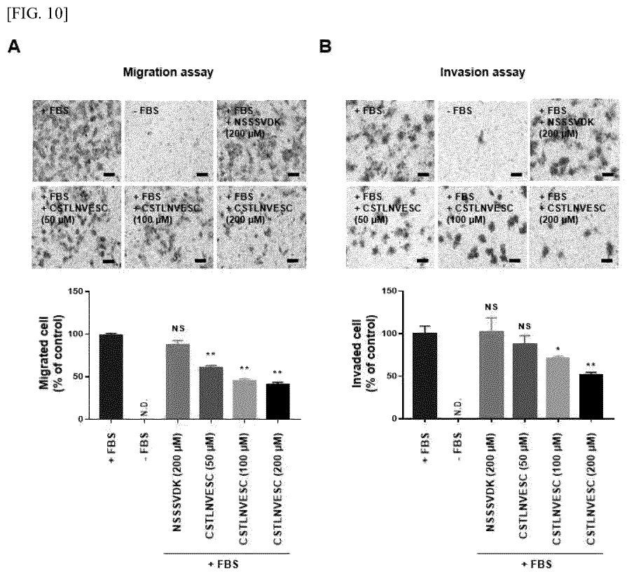

Trop2 is involved in EMT and metastasis, and to examine whether CSTLNVESC (SEQ ID NO: 1) peptide affects cell migration and invasion, trans-well chamber assays were performed. CSTLNVESC (SEQ ID NO: 1) peptide, but not the control peptide, inhibited the serum-induced migration and invasion of KKU-213 cells in a dose-dependent manner (FIG. 10).

### <Example 6> Tumor Homing of CSTLNVESC (SEQ ID NO: 1) Peptide

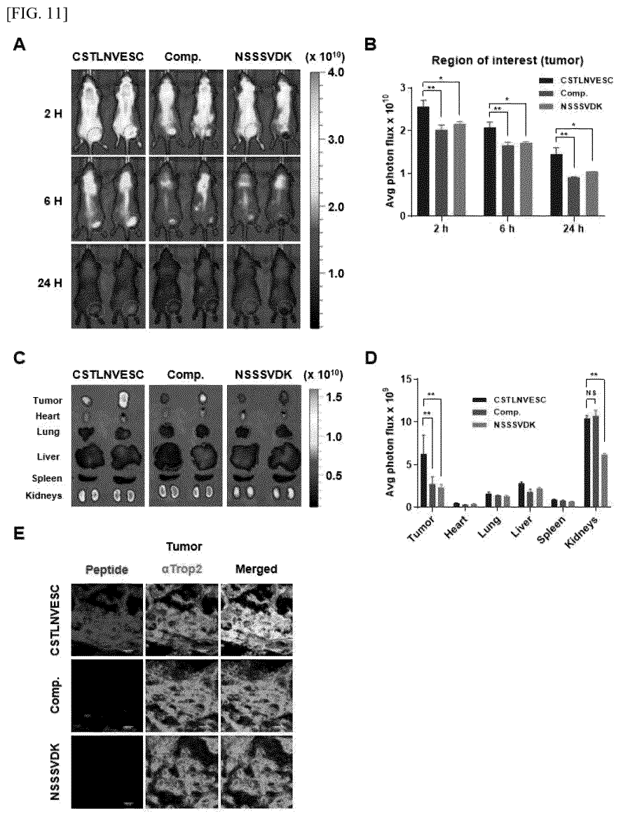

To examine the tumor homing of CSTLNVESC (SEQ ID NO: 1) peptide, the peptide was conjugated with Flamma 675 near-infrared dye and intravenously injected into mice bearing KKU-213 subcutaneous tumor. A whole-body imaging using the IVIS system at 2, 6, and 24 h after injection showed that CSTLNVESC (SEQ ID NO: 1)-Flamma 675 peptide homed to tumor more efficiently than the control peptide (FIG. 11A). In quantified data, the average photons of CSTLNVESC (SEQ ID NO: 1)-Flamma 675 peptide in tumor were higher than those of the control peptide until 24 h after injection (FIG. 11B). Ex vivo imaging (FIG. 11C) and quantification of average photons (FIG. 11D) in isolated organs further showed the accumulation of CSTLNVESC (SEQ ID NO: 1)-Flamma 675 peptide in tumor, while those of CSTLNVESC (SEQ ID NO: 1) and control peptides in other organs were similar except kidney. The high levels of photons in the kidney seem to be non-specific and due to urinary excretion of peptides. Immunofluorescence analysis using an anti-Trop2 antibody showed that CSTLNVESC (SEQ ID NO: 1) peptide, but not the control peptide, accumulated and co-localized with Trop2 in tumor tissues (FIG. 11E).

### <Example 7> Anti-Tumor Growth Activity of CSTLNVESC (SEQ ID NO: 1)-Targeted Cytotoxic Peptide

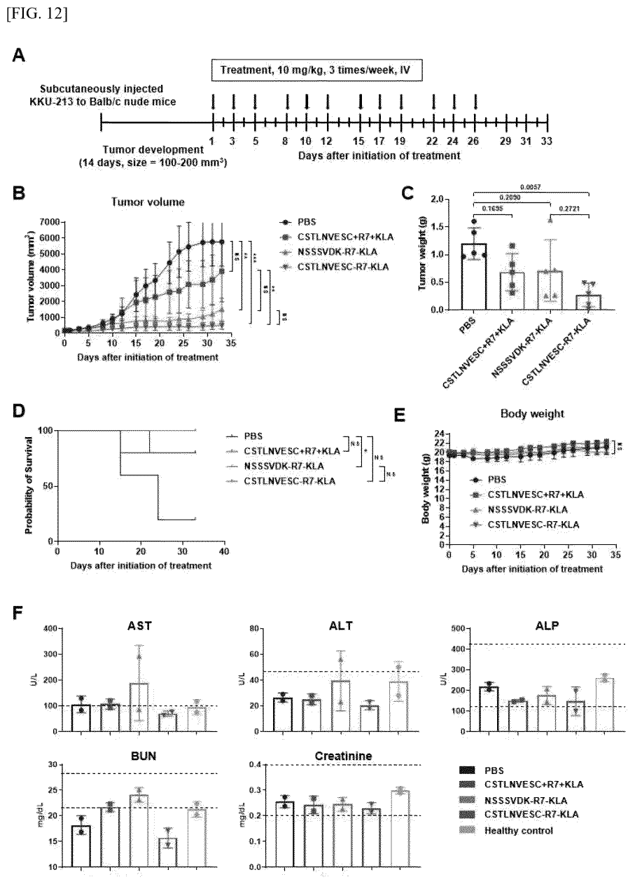

To exploit the use of CSTLNVESC (SEQ ID NO: 1) peptide for targeted cancer therapy, a Trop2-targeted cytotoxic peptide consisting of CSTLNVESC (SEQ ID NO: 1), RRRRRRR (SEQ ID NO: 2) (R7), and KLAKLAKKLAKLAK (SEQ ID NO: 3) (KLA) peptides was synthesized and named CSTLNVESC (SEQ ID NO: 1)-R7-KLA. R7 is a well-known cell-penetrating peptide that enhances the internalization of peptides and proteins into cells. KLA is a cytotoxic peptide that may cause damage to mitochondrial membrane of mammalian cells when introduced into cells and induce cell death. CSTLNVESC (SEQ ID NO: 1)-R7-KLA was intravenously injected into mice bearing KKU-213 tumor (10 mg/kg, three times per week, FIG. 12A). CSTLNVESC (SEQ ID NO: 1)-targeted R7-KLA peptide reduced tumor volume (FIG. 12B) and tumor weight (FIG. 12C) more efficiently than the control peptide-targeted R7-KLA or combined treatment of CSTLNVESC (SEQ ID NO: 1), R7, and KLA. On the other hand, all the treatments using peptides increased mouse survival more efficiently than the saline-treated control (FIG. 12D) and did not affect body weight (FIG. 12E). The levels of liver function enzymes (AST, ALT, and ALP) and kidney function markers (BUN and creatinine) were within normal ranges (FIG. 12F), suggesting no systemic side effects including toxicity to the liver and kidney, after treatment with CSTLNVESC (SEQ ID NO: 1)-R7-KLA.

Having described in detail a particular part of the present disclosure above, it will be apparent that, for a person skilled in the art, such a specific technique is only a preferred example embodiment, and the scope of the present disclosure is not limited thereby. Thus, the substantive scope of the present disclosure will be defined by the attached claims and their equivalents.

1. A peptide which has an amino acid sequence represented by SEQ ID NO: 1 and specifically binds to trophoblast antigen 2 (Trop2).

2-4. (canceled)

5. A method of diagnosing a cancer in a subject, comprising:
reacting the peptide of claim 1 with a tissue or cell obtained from the subject and
detecting the binding of the peptide to Trop2.

6. The method of claim 5, wherein the cancer is a cancer with a high expression level of Trop2.

7. The method of claim 6, wherein the cancer is any one or more selected from the group consisting of breast cancer, triple-negative breast cancer, lung cancer, pancreatic cancer, brain tumor, liver cancer, esophageal cancer, kidney cancer, colorectal cancer, rectal cancer, stomach cancer, thyroid cancer, bladder cancer, ovarian cancer, cholangiocarcinoma, bile duct cancer, gallbladder cancer, uterine cancer, cervical cancer, prostate cancer, head and neck cancer, laryngeal cancer, and oral cavity cancer.

8-12. (canceled)

13. A method of preventing or treating a cancer, comprising administering a pharmaceutical composition comprising a fusion peptide as an active ingredient in a subject,
wherein the fusion peptide comprises a peptide having an amino acid sequence represented by SEQ ID NO: 1 specifically binding to trophoblast antigen 2 (Trop2), a cell-permeable peptide having an amino acid sequence represented by SEQ ID NO: 2, and a proapoptotic peptide having an amino acid sequence represented by SEQ ID NO: 3.

14. The method of claim 13, wherein the cancer is a cancer with a high expression level of Trop2.

15. The method of claim 14, wherein the cancer is any one or more selected from the group consisting of breast cancer, triple-negative breast cancer, lung cancer, pancreatic cancer, brain tumor, liver cancer, esophageal cancer, kidney cancer, colorectal cancer, rectal cancer, stomach cancer, thyroid cancer, bladder cancer, ovarian cancer, cholangiocarcinoma, bile duct cancer, gallbladder cancer, uterine cancer, cervical cancer, prostate cancer, head and neck cancer, laryngeal cancer, and oral cavity cancer.

16-17. (canceled)

18. A pharmaceutical composition comprising the peptide of claim 1.


SEQUENCE LISTING (5 sequences)

SEQ ID NO: 1 - protein, 9 aa, synthetic construct
CSTLNVESC

SEQ ID NO: 2 - protein, 7 aa, synthetic construct
RRRRRRR

SEQ ID NO: 3 - protein, 14 aa, synthetic construct
KLAKLAKKLAKLAK

SEQ ID NO: 4 - DNA, 21 bp, synthetic construct
  source 1..21: mol_type = other DNA
cccgggatcgtttgcaagtaa

SEQ ID NO: 5 - DNA, 21 bp, synthetic construct
  source 1..21: mol_type = other DNA
ttgcgacattgtgaaggctta